(19) United States
(12) Plant Patent Application Publication
(10) Pub. No.: US 2003/0131392 P1
(43) Pub. Date: Jul. 10, 2003

(54) Petunia plant named 'SUNBELRE'

(76) Inventor: Yasuyuki Murakami, Gamo-gun (JP)

Correspondence Address: BURNS, DOANE, SWECKER & MATHIS, L.L.P., P.O. Box 1404, Alexandria, VA, 22313-1404, US

(21) Appl. No.: 10/327,030

(22) Filed: Dec. 24, 2002

(30) Foreign Application Priority Data
Dec. 28, 2001 (JP) 14311

(51) Int. Cl. A01H 5/00

(52) U.S. Cl. PLT/356

(57) ABSTRACT

Disclosed herein is a new and distinct variety of Petunia plant having a semi-decumbent growth habit. The Petunia plant has abundant branching particularly with respect to secondary branching, and great profusion of blooms with the entire plant remaining in bloom for a considerable period of time. The flowers are single and very small with the petals having a deep red color. The base color of the corolla throat is vivid greenish yellow and the outside of the corolla tube is olive gray. The plant exhibits high resistance to rain, heat, drought and diseases such as Powdery mildew.

### BACKGROUND OF THE VARIETY

The Petunia is a very popular plant that is used for flower bedding and potting in the summer season. There are only a few Petunia developed series of small flower petunias, such as, ‘SUNTORY SP-R’ (U.S. Plant Pat. No. 9557), ‘SUNBERUBU’ (U.S. Plant Pat. No. 9754), ‘SUNBELCHIPI’ (U.S. Plant Pat. No. 10,355) and ‘SUNBELUKI’ (U.S. Plant Pat. No. 11,558) that are of the semi-decumbent type, a medium plant height, with abundant branching, and a high resistance to heat and rain and disease. Also there are only a few varieties having a great profusion of flowers, a deep purplish pink˜strong purplish pink flower color, a very small flower size and a high resistance to rain, heat, and diseases. Accordingly, this invention was aimed at obtaining a new variety having deep red petals, and very small flowers combined with the above features.

The new variety of Petunia plant according to this invention originated from crossing of ‘SUNBELCHIPI’ (female) variety (U.S. Plant Pat. No. 10355) and a Petunia plant variety called ‘R5’ (male).

First of all, a crossing was conducted with the ‘SUNBELCHIPI’ variety as female parent and a seedling of petunia plant variety ‘R5’ as pollen parent in May 1997 at the Omi R&D Center of SUNTORY Ltd., located at 863-1, Aza-Iketani, Omori-cho, Youkaichi-shi, Shiga-ken, Japan. From that crossing 50 seedlings were obtained and 5 seedlings were selected in view of their semi-erect growth habit and deep purplish pink colored petals by the end of September 1998. These 5 seedlings were grown and were tested during potting and bedding trials. Only one plant of the present invention was selected in September 1999. The selected plant was propagated by cutting and then grown in pots as a trial in the greenhouses and the field at the Omi R&D Center from April to November 2000. The botanical characteristics of the finally selected plant were examined, using similar variety ‘SUNBELCHIPI’ (U.S. Plant Pat. No. 10355) for comparison. As a result, it was concluded that this new Petunia is distinguishable from any other variety, whose existence is known to us, and is uniform and stable in its characteristics, and then this new variety of Petunia plant was named ‘SUNBELRE’.

In the following description, the color information is in accordance with The R.H.S. Color Chart of The Royal Horticultural Society, London, England. A color chart based on The Japan Color Standard for Horticultural Plants (J.H.S. Color Chart) is also added for reference.

The ‘SUNBELCHIPI’, used as female parent and a comparison in the obtaining of this new variety ‘SUNBELRE’, is a bred variety previously bred by us and filed in Japan (Seeds and Seedlings law Registration No. 7232) and U.S.A. (U.S. Plant Pat. No. 10355). The main botanical characteristics thereof are as follows:

Plant:

Growth habit.—Semi-decumbent.

Plant height.—15-16 cm.

Spreading area of plant.—The stem extends to length of 11-13 cm from the base.

Blooming period.—April to late October in the southern Kanto area, Japan. The plant shape does not change throughout this period.

Stem:

Thickness.—2.3 mm.

Pubescence.—Present.

Branching.—Abundant with the superior branching propensity, especially secondary branches are.

Length of internode.—1.4 cm.

Leaf:

Shape.—Lanceolate.

Length.—5.1 cm.

Width.—1.6 cm.

Color.—Dark yellow green (R.H.S. 146A, J.H.S. 3508).

Thickness.—0.2-0.4 mm.

Pubescence.—Sparse.

Leaf attaching angle to stem.—Slantly upward to horizontal.

Flower:

Facing direction.—Horizontal.

Type.—Single.

Shape.—Funnel-shape, with five-fissured limb.

Shape of petal tip.—Round.

Waving of petal.—Weak

Lobation of petal.—Shallow.

Diameter.—2.8-2.9 cm.

Color.—Petal: Vivid purplish red (R.H.S. 57C, J.H.S. 9707). Bottom color of corolla throat: Strong yellow (R.H.S. 9A, J.H.S. 2513). Outside color of corolla tube: Light greenish yellow (R.H.S. 5C, J.H.S. 2904).

Reproductive organs.—1 normal pistil and 5 normal stamens.

Fertility.—Fertile, but self-incompatible.

Peduncle.—0.7-0.8 mm in thickness, and 1.9 cm in length.

Physiological and ecological characteristics.—High resistance to heat, rain, drought and diseases such as Powdery mildew. The resistance to heat and rain is very strong.

Blooming:

A bloom commonly lasts approximately 10 days on the plant. Pinching is not necessary to ensure continuous blooming; however, it does tend to enhance bloom production.

The ‘R5’, used as male parent in the obtaining of this new variety ‘SUNBELRE’, are a bred variety bred by us and not filed in Japan nor any other country. ‘R5’ had been grown at the Omi R&D Center of SUNTORY Ltd., located at 863-1, Aza-Iketani, Omori-cho, Youkaichi-shi, Shiga-ken, Japan. The main botanical characteristics thereof are as follows:

Plant:

Growth habit.—Semi-erect.

Plant height.—20 cm.

Spreading area of plant.—The stem extends to length of 5 cm from the base.

Blooming period.—April to October in the southern Kanto area, Japan. The plant shape does not change throughout this period.

Stem:

Thickness.—2.4 mm.

Pubescence.—Sparse.

Branching.—Abundant with the superior branching propensity, especially secondary branches are.

Length of internode.—1.3 cm.

Leaf:

Shape.—Lanceolate.

Length.—3.4 cm.

Width.—1.1 cm.

Color.—Dark Olive green (R.H.S. 137A, J.H.S. 3707).

Thickness.—0.5 mm.

Pubescence.—Sparse.

Leaf attaching angle to stem.—Horizontal to droopy.

Flower:

Facing direction.—Upward to Slantly upward.

Type.—Single.

Shape.—Funnel-shape, with five-fissured limb.

Shape of petal tip.—Round.

Waving of petal.—Weak.

Lobation of petal.—Shallow.

Diameter.—2.9 cm.

Color.—Petal: Vivid reddish orange (R.H.S. 33A, J.H.S. 0705)ground color with vivid red (R.H.S. 44A, J.H.S. 0707) streak, grayish red (R.H.S. 177A, J.H.S. 0419) center margin. The bottom color of corolla throat: Vivid yellow (R.H.S. 15A, J.H.S. 2205). The outside color of corolla tube: Brilliant yellow (R.H.S. 15C, J.H.S. 2204).

Reproductive organs.—1 normal pistil and 5 normal stamens.

Fertility.—Fertile, but self-incompatible.

Peduncle.—0.8 mm in thickness, and 2.0 cm in length.

Physiological and ecological characteristics.—High resistance to heat, rain, drought and diseases such as Powdery mildew. The resistance to heat and rain is very strong.

Blooming:

A bloom commonly lasts approximately 10 days on the plant. Pinching is not necessary to ensure continuous blooming; however, it does tend to enhance bloom production.

### SUMMARY OF THE VARIETY

This new variety of Petunia plant ‘SUNBELRE’ has a semi-decumbent growth habit with abundant branching, with single and very small deep red color flowers and forms a great profusion of blooms with the whole plant remaining in bloom for a considerable period of time. The plant has high tolerances to cold and heat, high resistance to pests and diseases, particularly powdery mildew, and high resistance to rain.

### BRIEF DESCRIPTION OF THE PHOTOGRAPHS

FIG. 1 is a photograph of flowers and leaves of the new variety of Petunia plant ‘SUNBELRE’.

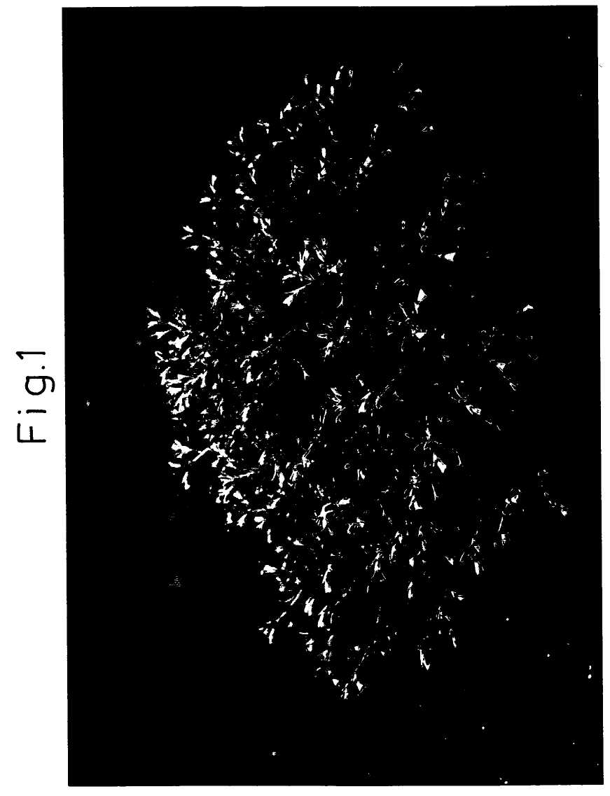

FIG. 2 is a photograph of flowers of the new variety of petunia plant ‘SUNBELRE’

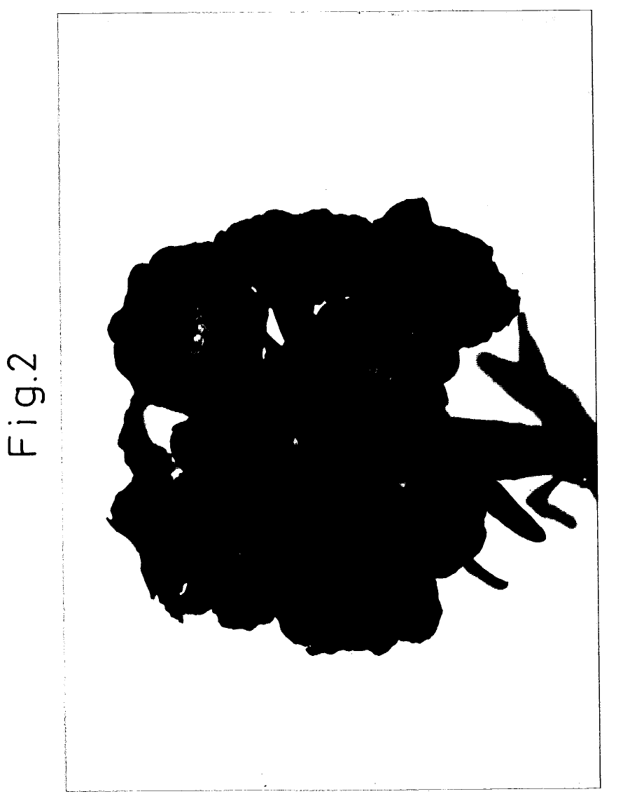

### DESCRIPTION OF THE NEW VARIETY

The botanical characteristics of the new and distinct variety of Petunia plant named ‘SUNBELRE’ are as follows when observing during October at Yokaichi-shi, Shiga-ken, Japan young plants that had been reproduced by cuttings in June.

Plant:

Growth habit.—Semi-decumbent.

Plant height.—14 cm.

Spreading area of plant.—The stem extends to length of 8 cm from the base.

Blooming period.—April to late October in the southern Kanto area, Japan. The plant shape does not change throughout this period.

Stem:

Thickness.—2.4 mm.

Pubescence.—Sparse.

Branching.—Abundant with the superior branching propensity, especially secondary branches are.

Length of internode.—1.3 cm.

Leaf:

Shape.—Lanceolate.

Length.—5.2 cm.

Width.—1.2 cm.

Color.—Moderate olive green (R.H.S. 147A, J.H.S. 3509).

Thickness.—0.6 mm.

Pubescence.—Sparse.

Leaf attaching angle to stem.—Slanted upward to horizontal.

Flower:

Facing direction.—Horizontal.

Type.—Single.

Shape.—Funnel-shape, with five-fissured limb.

Shape of petal tip.—Round.

Waving of petal.—Weak.

Lobation of petal.—Shallow.

Diameter.—2.8 cm.

Color.—Petal: Deep red (R.H.S. 46A, J.H.S. 0408). Bottom color of corolla throat: Vivid greenish yellow (R.H.S. 153D, J.H.S. 2706). Outside color of corolla tube: Brilliant greenish yellow (R.H.S. 8A, J.H.S. 2704).

Reproductive organs.—1 normal pistil and 5 normal stamens.

Fertility.—Fertile, but self-incompatible.

Peduncle.—0.8 mm in thickness, and 1.9 cm in length.

Physiological and ecological characteristics.—High resistance to heat, rain, drought and diseases such as Powdery mildew. The resistance to heat and rain is very strong.

Blooming:

A bloom commonly lasts approximately 10 days on the plant. Pinching is not necessary to ensure continuous blooming; however, it does tend to enhance bloom production.

This new variety of Petunia plant is most suitable for flower bedding and potting, particularly in hanging pots or planters, and is excellent for use as ground cover. Pinching of old blossoms will enhance the formation of new blossoms.

I claim:

1. A new and distinct variety of Petunia plant, substantially as herein illustrated and described, characterized particularly as to novelty by (A) being semi-decumbent growth habit, (B) a great profusion of blooms with the entire plant remaining in bloom for a considerable period of time, (C) flowers are single and very small, the petals have a deep red color, and (D) a high resistance to rain, heat, drought and pests.
